(19) United States
(12) Patent Application Publication
(10) Pub. No.: US 2022/0347286 A1
(43) Pub. Date: Nov. 3, 2022

(54) INACTIVATED VACCINE FOR CHIKUNGUNYA VIRUS

(71) Applicant: The Government of the United States, as Represented by the Secretary of the Army, Fort Detrick, MD (US)

(72) Inventors: Stephen J. THOMAS, Rockville, MD (US); Kenneth H. ECKELS, Rockville, MD (US); Joseph R. PUTNAK, Silver Spring, MD (US); Richard G. JARMAN, III, Frederick, MD (US); Rafael Antonio DE LA BARRERA, Washington, DC (US)

(21) Appl. No.: 17/851,813

(22) Filed: Jun. 28, 2022

Related U.S. Application Data
(62) Division of application No. 16/069,073, filed on Jul. 10, 2018.
(60) Provisional application No. 62/278,166, filed on Jan. 13, 2016.

(51) Int. Cl. A61K 39/12; C12N 7/00

(52) U.S. Cl. CPC A61K 39/12; C12N 7/00; C12N 2770/36134; C12N 2770/36163; C12N 2770/36164; Y02A 50/30; A61K 2039/5254

(57) ABSTRACT

The disclosure generally provides a purified inactivated chikungunya virus (CHIKV), methods for producing the purified inactivated CHIKV, immunogenic compositions and vaccines comprising the purified inactivated CHIKV and methods for the prevention and/or treatment of infection by CHIKV.

### CROSS REFERENCE TO RELATED APPLICATIONS

This application is a divisional of U.S. application Ser. No. 16/069,073, filed 10 Jul. 2018, which claims priority to International Application No. PCT/US2017/013417, filed 13 Jan. 2017, which claims priority to expired U.S. Provisional Application No. 62/278,166, filed 13 Jan. 2016. The content of each of these applications is herein incorporated by reference in their entirety.

### FIELD

The disclosure relates to immunogenic compositions, vaccines, and methods for immunization and protection (e.g., prophylaxis) against chikungunya virus (CHIKV) infection, associated diseases, and clinical conditions. More particularly, the disclosure provides a pure, inactivated composition comprising virus that is re-derived from an attenuated CHIKV strain, and which confers an antibody titer sufficient for broad-based sero-protection against all strains of CHIKV.

### BACKGROUND

Chikungunya virus (CHIKV) is a small enveloped RNA alphavirus of the family Togaviridae. Typically it is transmitted to humans by Aedes sp. mosquitoes. Phylogenetic analyses have been used to characterize and identify three viral genotypes, including West African, East/Central/South African (ECSA), and Asian. CHIKV infections can cause acute fever and severe arthritis-like joint pain (arthralgia), which may persist from weeks, months, or years after convalescence. Other reported symptoms and conditions include fatigue, headache, nausea, vomiting, muscle pain, rash, and in some cases may be partially responsible for death. Less common manifestations of disease may result in gastrointestinal, eye, neurologic, and cardiac complications. Widespread epidemics have been reported in Africa, the Middle East, Europe, India, and Southeast Asia. More recently, significant outbreaks have occurred in the Americas, particularly in South America and the Caribbean. Since 2006 more than 200 cases have been reported in the continental United States in travelers returning from other countries, and in 2014 the Centers for Disease Control and Prevention (CDC) reported the first case of CHIKV contracted in the United States in Florida. In September 2014, the CDC had reported 7 confirmed cases of locally acquired CHIKV in the United States. It is estimated that the population at risk of contracting CHIKV infection is greater than 3 billion people.

Currently, there are no approved or licensed vaccines to prevent CHIKV infection or disease, leaving sustained and rigorous control of the mosquito vector and personal protective measures as the only methods of reducing the burden of disease. Some DNA-based and live attenuated CHIKV vaccine candidates have been tested in Phase I clinical trials. Nevertheless, the live attenuated vaccines carry intrinsic concerns with side effects, some of which may arise from potential insufficient and/or instable attenuation, and the DNA vaccines have exhibited weak immunogenicity; none have been proven effective n clinical endpoint trials. There remains an acute need for a vaccine capable of inducing an immune response profile which will protect the recipient from the spectrum of disease resulting from CHIKV infection, without risking the potential for reactogenicity.

### SUMMARY OF THE DISCLOSURE

The disclosure generally provides a purified, inactivated CHIKV as well as compositions and vaccines comprising the inactivated CHIKV, methods for generating the same, and methods of generating an immune response in a subject at risk of infection and/or in need of preventative treatment.

In an aspect, the disclosure provides a vaccine that immunizes against CHIKV. In some embodiments, the vaccine protects against disease prior CHIKV exposure and infection. In some embodiments, the vaccine may alleviate disease and clinical symptoms associated with CHIKV following CHIKV exposure. In some aspects, the disclosure provides a vaccine that is suitable for rapid immunization with the potential to break the cycle of viral transmission at the individual and population levels.

In an aspect, the disclosure relates to a composition comprising a purified inactivated CHIKV preparation. In some embodiments, the composition may comprise a vaccine.

In a further aspect, the disclosure provides a method for generating a purified inactivated CHIKV comprising inoculating a cell culture with a CHIKV strain; propagating the virus in the inoculated cell culture; harvesting and isolating virus fluids from the inoculated cell culture to prepare a CHIKV concentrate; purifying the CHIKV concentrate; inactivating the purified CHIKV; and recovering the inactivated purified CHIKV. In some embodiments, inactivation comprises contacting the CHIKV with a chemical inactivating agent. In further embodiments, the chemical inactivating agent comprises formalin or beta-propiolactone. In some embodiments, die propagating may comprise one, two, three, four, or five cell passages. In some embodiments, the disclosure provides a purified inactivated CHIKV generated by the method.

In some embodiments of the above aspects, the CHIKV may be derived from any publicly available CHIKV strain or publicly available CHIKV sequence. In embodiments, the CHIKV may be derived from a live attenuated CHIKV strain. In some embodiments, the CHIKV may be derived from a CHIKV strain that is genetically modified to create an attenuated CHIKV strain. In some embodiments the CHIKV may be generated in vitro using PCR transcription of infective or attenuated cDNA CHIKV clones. In some embodiments the CHIKV may be the CHIKV 181/clone 25 strain. In some embodiments the CHIKV may derived from the CHIKV 181/clone 25 strain. In some embodiments the CHIKV may be a strain that is deposited as ATCC Deposit No. PTA-126983.

In a further aspect, the disclosure relates to a method for immunizing a mammal against a CHIKV, wherein the method comprises administering to the mammal an amount of the vaccine disclosed herein to achieve effective immunization against CHIKV. In some embodiments, the method is effective at eliciting a protective immune response in a human against CHIKV. In some embodiments, the administration is via a route selected from intramuscular injection, intradermal injection, subcutaneous injection, intravenous injection, oral, or intranasal inoculation.

In a further aspect, the disclosure relates to the use of the purified inactivated CHIKV disclosed herein in the production of a medicament for the prophylaxis of CHIKV infection.

In a further aspect, the disclosure relates to the use of the purified inactivated CHIKV disclosed herein in the production of a medicament for the treatment of CHIKV infection.

Other aspects will be apparent to one of skill in the art upon review of the description and exemplary aspects and embodiments that follow.

### BRIEF DESCRIPTION OF THE DRAWINGS

For the purpose of illustrating the disclosure, there are depicted in the drawings certain features of the aspects and embodiments of the disclosure. However, the disclosure is not limited to the precise arrangements and instrumentalities of the aspects depicted in the drawings.

FIG. 1 shows the A280 value for CHIKV fractions from the CaptoCore700 column purification. White bars are for a column nm at 2.0 mL/min and the gray bars are for a column nm at 1.2 mL/min. Column fractions 2-5 from the 2.0 mL/min trial were pooled for inactivation while fractions 2-6 from the 1.2 mL/min were pooled for inactivation (indicated by arrows).

FIG. 2A-2B depicts the inactivation of column purified CHIKV using BPL (FIG. 2A) and formalin (FIG. 2B) as a function of time.

FIG. 3 illustrates a SDS-PAGE silver stain of CHIKV (181-25) CaptoCore700 Purified-Inactivated Lot 1925. Lane 1=molecular weight markers (in kDa); Lane 2=Clarified Viral Fluids Post Benzonase (27 μg/ml); Lane 3=Final Bulk BPL Inactivated (7 μg/m); Lane 4=Final Bulk Formalin Inactivated (14 μg/ml); Lane 5=Purified Retentate-Pooled Fractions (18 μg/ml). As shown by the arrows, clarified viral fluids following benzonase were used to generate the pooled fractions of purified retentate (lane 5), which was used to generate each of the final bulk inactivated virus (BPL in lane 3 formalin in lane 4).

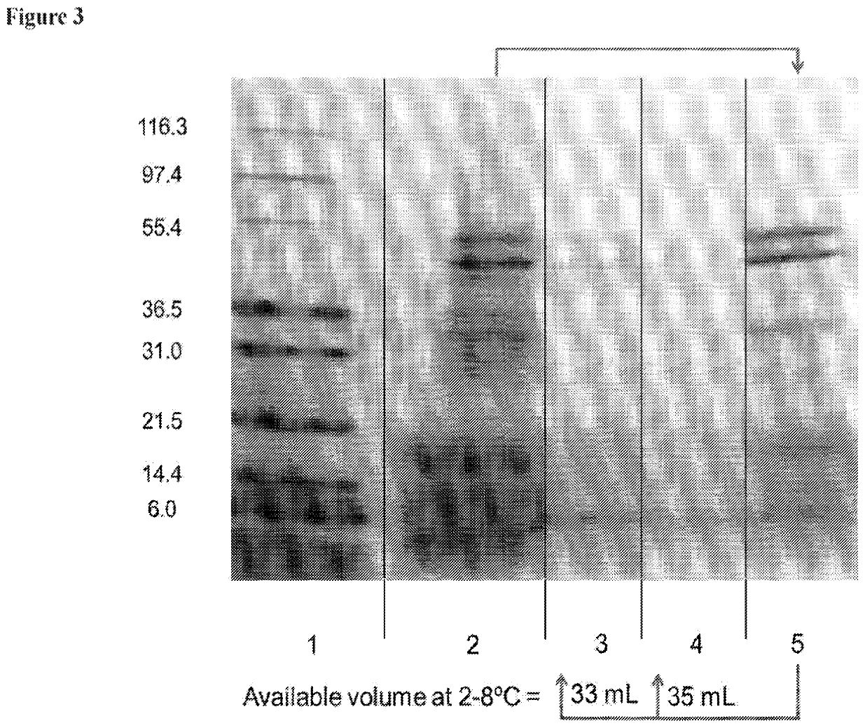

FIG. 4 provides a generalized process flow chart for inactivated CHIKV vaccine production.

### DETAILED DESCRIPTION

Before continuing to describe various aspects aid embodiments in further detail, it is to be understood that this disclosure is not limited to specific compositions or process steps and may vary. As used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this invention is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this invention.

A “vaccine” as referred herein is defined as a pharmaceutical or therapeutic composition used to inoculate an animal in order to immunize the animal against infection by an organism, such as CHIKV. Vaccines typically comprise one or more antigens derived from one or moue organisms (CHIKV) which on administration to an animal will stimulate active immunity and protect that animal against infection with these or related pathogenic organisms.

“Purified” as used herein in association with the inactivated virus or vaccine means that the CHIKV virus or viral particles are separated from non-viral molecules such as, for example, host cell proteins and nucleic acids.

As used herein, “immunogenic” has the same meaning as is well known in the art, as generally relating to a substance that can induce an immune response or enable a substance to induce an immune response. In certain aspects and embodiments, the disclosure provides compositions, such as vaccines, that comprise a purified and inactivated CHIKV and an adjuvant. Such immunogenic compositions can be used in methods, or in medicaments, useful for inducing an immune response against CHIKV (e.g., production of antibodies against CHIKV) when administered to a subject.

The inventors have developed immunogenic compositions, including a purified inactivated vaccine (PIV), effective in immunizing a subject and preventing disease and clinical symptoms associated with or caused by CHIKV infection. Immunogenic compositions and vaccines comprising the inactivated virus can provide for a global vaccine protecting the recipient from disease caused by any CHIKV strain from any part of the world. Other purified inactivated viruses have been successfully employed as vaccines against other viral agents including, for example, Japanese encephalitis (JE), and Tick Borne Encephalitis, and have been shown experimentally to have promising results in other diseases such as dengue (DENV) and yellow fever (YFV). As understood in the art, each of the pathogens associated with such inactivated viruses have distinct diseases and clinical symptoms associated with it, and success may not be predictive of success against any of the other pathogens. Furthermore with regard to CHIKV, prior to the inventors' development of the purified and inactivated CHIKV disclosed herein, questions remain as to whether a viable and effective CHIKV vaccine candidate could be developed using safer inactivated virus technology rather than live-attenuated technology (see, e.g., Roy, C. J., et al., J. Infec. Dis, 209(12): 1891-9 (Jun. 15, 2014). In a general sense, the inactivated CHIKV disclosed herein is prepared by taking a live CHIKV and inactivating it using a process such as disclosed herein. Once the inactivated virus is obtained, it can be used to prepare compositions, such as vaccines, that are effective to generate a prophylactic immune response against CHIKV infection. Additional embodiments are contemplated wherein the compositions, including vaccines, are effective to generate a therapeutic immune response against CHIKV infection.

Despite the existing skepticism regarding immunogenic efficacy of an inactivated CHIKV, the inventors successfully produced a PIV CHIKV that confers protection and takes advantage of the many benefits the technology provides relative to other types of immunogenic products, and particularly attenuated live viruses. Such advantages of a PIV include an additional margin of safety by virtue of the absence of genetic reversion to a virulent, wild type virus, potentially lower acute reactogenicity following vaccination, reduced potential for causing prolonged symptoms in joint spaces, rapid immunization timelines, potential to co-administer with other vaccines, and the like. Thus, the vaccine comprising an inactivated CHIKV as described herein can have an excellent safety profile with no risk for reversion and the potential to confer protective immunity more quickly than live attenuated vaccines without their undesirable side effects. Not only are the inactivated CHIKV vaccines more stable and safer than live CHIKV vaccines, they are usually easier to store and transport as they do not require refrigeration. Further such compositions can be easily stored and transported in a freeze-dried form, which provides for greater accessibility to people in developing countries.

In an aspect, the disclosure provides an inactivated CHIKV strain that is suitable for inducing an immune response in a subject.

In another aspect, the disclosure provides a method for producing a purified inactivated CHIKV strain that is suitable for inducing an immune response in a subject

In embodiments of these aspects, the immune response may be induced in a virus naive subject. In other embodiments, the immune response may be induced in a subject who has been exposed to a wild type CHIKV.

In some aspects, the disclosure provides a method for producing the inactivated CHIKV. In some embodiments the method comprises re-derivation of the vaccine production seed by RNA transfection. Such embodiments can provide for a composition/vaccine that is free from any contaminating adventitious agents and provides an additional margin of safety. Nevertheless, the inactivated virus and immunogenic compositions can be produced by any method generally known in the art. In embodiments, the CHIKV may be rendered non-infectious by killing/inactivating the virus by heat, gamma irradiation, UV light, or by a chemical agent, such as formalin or beta-propiolactone (BPL) among others under conditions that retain high immunogenicity of the vaccine preparation. In some embodiments the inactivation can comprise one or more chemical inactivating agents including, but not limited to, formalin, beta-propiolactone, glutaraldehyde, N-acetylethylenimine, binary ethylenimine, tertiary ethylenimine, ascorbic acid, caprylic acid, psolarens, detergents including non-ionic detergents, and the like. In such embodiments, the chemical inactivating agent is added to a virus suspension in an amount effective to inactivate the virus. In some embodiments, the inactivation is performed at a temperature from about 4° C. to about 22° C. In some preferred embodiments, the inactivated virus is produced by a method as described herein.

Purification of the inactivated CHIKV may be performed by physical or chemical techniques or any combinations thereof that are routinely used in the art. Physical methods utilize the physical properties of the virus such as density, size, mass, sedimentation coefficient, and the like, and include but are not limited to, ultracentrifugation, density gradient centrifugation, ultrafiltration, size-exclusion chromatography, and the like. Chemical purification can employ methods such as adsorption/desorption through chemical or physiochemical reactions such as ion exchange chromatography, affinity chromatography, hydrophobic interaction chromatography, hydroxyapatite matrix, precipitation with inorganic salts such as ammonium sulfate, and the like.

As disclosed herein, the disclosure provides a purified, inactivated vaccine to protect against disease caused by CHIKV. In some embodiments, the inactivated CHIKV is produced from a starting material (reference CHIKV). The reference CHIKV may be selected from any CHIKV strain that has been identified, characterized, and/or isolated as known in the art. In some embodiments the reference CHIKV is a live CHIKV isolate. In some preferred embodiments, the reference CHIKV is an attenuated CHIKV strain. In some of the preferred embodiments, the attenuated CHIKV strain may be adapted to grow in Vero cells by 2-3 cell serial passages at a low multiplicity of infection (MOI). In such embodiments, the passaged CHIKV strain may be re-derived by RNA transfection using any standard method known in the an, in a suitable cell line such as, for example, Vero cells that have been certified for vaccine production. In such embodiments the re-derived strain may have a reduced risk of containing any potential adventitious agents that may otherwise induce an adverse event or side effect when administered to a subject. The purified and inactivated re-derived virus may be used to produce a vaccine master seed lot and/or a working seed lot. Some non-limiting embodiments of CHIKV that may be used in the compositions and methods herein include strain AF15561 (GenBank EF452493), 181/clone 25 (GenBank accession no. 1.37661, also identified as TSI_GSD-218), La Reunion (LR) strain (GenBank EU224268.1), or other strains available at the NCBI website (GenBank). In some preferred embodiments, the CHIKV is strain 181/clone 25 strain, or is derived from strain 181/clone 25 according to the methods disclosed herein. In some embodiments the reference CHIKV may comprise the strain deposited as ATCC Deposit No. PTA-126983.

In certain aspects, the disclosure relates to the use of an attenuated CHIKV strain to produce the CHIKV purified, inactivated vaccine (PIV). In embodiments of this aspect the use encompasses the preparation of a medicament and/or a prophylactic composition for use against CHIKV infection.

As discussed above, the disclosure provides a method for producing an inactivated CHIKV virus that may be used for production of vaccine lots. In such aspects, the method may comprise infection of a suitable cell line for vaccine manufacture. For example, in some embodiments, the method may comprise certified Vero cells grown in roller bottles, cell factories, or suspension cultures that are infected with the CHIKV working seed at a suitable MOI (e.g., 0.1 to 0.001). After infection the cell culture fluids containing the virus are harvested based on the development of cytopathology (e.g., 50% or more cells showing cytopathic effects, CPE) and/or viral antigen yields measured by a suitable assay such as virus hemagglutination (HA) or ELISA. Depending upon the infection time course and the amount of cytopathology the virus may also be harvested continuously or at intervals throughout the infection cycle with replacement of removed culture medium. The collected bulk supernatant harvests are pooled and concentrated approximately 80- to 100-fold by a suitable method, (e.g., tangential flow ultrafiltration using an appropriate membrane pore size to retain the virus and remove small MW contaminants). The virus concentrate can be subjected to a treatment that removes residual host cell nucleic acids and contaminating cellular proteins such as, for example, Benzonase treatment or protamine sulfate precipitation. The concentrated, treated virus pool may then be purified by a suitable method such as density gradient centrifugation, rate zonal centrifugation, continuous flow centrifugation, or column chromatographically, and the virus peak fractions may be identified by optical density (OD), HA or ELISA, and pooled. The purified virus concentrate can be quantified for protein, infectivity and viral and host cell antigen content and host nucleic acids.

Inactivation of the purified virus can be performed by any suitable method that preserves viral antigenicity such as formalin or beta-propriolactone (BPL). For example, inactivation with formalin can be performed at 4-22° C. for a time sufficient to achieve complete inactivation of infectivity, considering also the recommended three-fold safety margin since formalin inactivation is non-linear. Optional filtration through a 0.22 um filter may be performed, and the filtered material transferred to a fresh container at 48 his b remove virus aggregates resistant to inactivation. In some embodiments, BPL, which may be faster and exhibit more linear kinetics, may be used for inactivation. Typically, the inactivating agent is neutralized (e.g., with sodium bisulfite in the case of formalin) or removed by diafiltration.

Bulk vaccines may be tested for sterility, protein, antigen and nucleic acid content using established assays. Residual infectivity can be assayed by inoculation of approximately 5% of the lot volume onto Vero cell cultures, or another suitable cell line, followed by incubation for a sufficient time to amplify any residual infectious virus present, which can then be detected by FFA directly on the cells or by plaque assay of the culture supernatants. Following inactivation the bulk vaccines can be mixed with suitable excipients and/or stabilizers and stored frozen (e.g., −20° C. to −80° C. prior to formulation). Inactivated CHIKV bulk may be diluted to a protein concentration that is suitable for an immunizing dose in a subject (e.g., a mammal such as a human). The final, vialed vaccine may be tested for purity, identity, osmolality, endotoxin, and sterility by various, standardized assays generally known in the art.

As discussed below, any one of the known animal models that have been developed to study the virulence of CHIKV may be used to determine the infectivity/virulence/efficacy of the CHIKV strains and/or vaccines prepared by the methods disclosed herein. A number of mouse models have been developed and are accepted as models of CHIKV infection and disease in humans. See, fir example, Gorchakov, R., et al., J. Virol., June 2012; 86(11): 6084-6096; Coudere, T., et al., PLoS Pathog., 2008; 4:e29; Gardner, J., et al., J. Virol., 2010; 84; 8021-8032; Morrison, T. E., et al., Am. J. Pathol., 2011; 1714:32-40; Ziegler, S. A., et al., Am. J. Trop. Med. Hyg., 2008; 79:133-139, which discuss various mouse models including infant outbred CD1 mice and A129 inbred mice. Efficacy and/or virulence may also be tested according to well-known methods in higher vertebrates such as non-human primates (e.g., cynomolgus macaques) using randomly assigned cohorts for vaccination with candidate compositions and sham (e.g., saline) compositions, and subsequent challenge with wild type CHIKV. Results can be determined using analysis and assays performed on samples taken from blood and tissue collected after challenge.

In some embodiments, immunogenic potency of bulk vaccine lots and the final formulation can be tested by administering the vaccines to mice. Typically, groups often 5-6 week-old, female, Swiss-ICR mice receive serially graded doses ranging from about one nanogram to one microgram of vaccine, as required to reach an endpoint, in a 0.1 ml intramuscular or subcutaneous dose. A corresponding control group receives saline or saline plus adjuvant, as appropriate. Mice are typically boosted once; this can be done on day 14 or 28 after priming, and then blood is collected one to two weeks later. The sera from individual mice awe assayed for virus neutralizing antibodies and the vaccine median immunizing dose (ID50) is calculated. In this way vaccine potency may be monitored periodically.

In embodiments, an animal efficacy study is designed to demonstrate that the vaccine induces an effective immune response including virus neutralizing antibodies and protection against a live virus challenge in comparison to a placebo control. Also, the animals are observed during the course of the study for any adverse effects. This testing is necessary before a vaccine can progress to a clinical trial Typically, such experiments are best performed in a non-human primate infection model (e.g., rhesus macaques) with the primary endpoints being the measurement of virus neutralizing antibodies after vaccination and the measurement of protection against challenge with an attenuated or wild type CHIKV strain. Protection can be assessed by a disease surrogate such as circulating virus (viremia) after virus challenge, which allows for the use of an attenuated challenge virus strain under BSL-2. Various vaccine doses and immunization schedules can also be tested in the experiment. Group sizes of 5 to 10 are suitable for a pilot study. For example, using Fisher's Exact Test with alpha=0.05 (2-sided) and n=5 animals per group: for 100% vs. 0%, or 100% vs. 5%, the power is about 80%. Responses can be compared and contrasted for individual animals and among groups using standard statistical methods. For example, log-transformed antibody and viremia titers can be analyzed by ANOVA. Fisher's exact test can be used to compare rates of seroconversion to each virus antigen and viremia rates among vaccine groups and placebo controls. A one-way analysis of variance with a contrast test for trend may be used to assess differences in antibody or viremia titers among groups. To stabilize the variance the analysis is conducted on the logs of the quantified responses. A test for trend using the logistic model can be used to assess differences in the proportion of seroconverters.

Reactogenicity of the vaccines disclosed herein may be monitored and evaluated as may be necessary. A reactogenicity event is typically identified as an adverse event that is commonly known to occur for the candidate therapeutic/prophylactic product being studied. Typically, such events are collected in a standard, systematic format using a graded scale based on functional assessment or magnitude of reaction. This helps to provide a risk profile of the candidate product and a defined listing of expected (or unexpected) adverse events, and whether such events are local or systemic events.

The disclosed methods and compositions may be applied to any CHIKV strain as discussed above. In some embodiments the inactivated CHIKV is derived by a method as disclosed herein from live attenuated CHIKV strain 181/25. In some embodiments the CHIKV is derived from the strain deposited as ATCC Deposit No. PTA-126983. The vaccines disclosed herein may offer good immune protection against multiple (heterologous) strains of

CHIKV in addition to the particular CHIKV strain(s) used in production of the vaccine. The CHIKV isolates may exhibit broad neutralizing activity and may cross-neutralize different genotypes/genotypic variants/strains of CHIKV.

The purified and inactivated CHIKV vaccine Is prepared for administration b mammals, suitably humans, mice, rats or rabbits, by methods known in the art, which can include filtering to sterilize the solution, diluting the solution, adding an adjuvant and stabilizing the solution.

The vaccines disclosed herein may be administered to a human or animal by a number of routes, including but not limited to, for example, parenterally (e.g. intramuscularly, transdermally), intranasally, orally, topically, or other mutes know by one skilled in the art. The term parenteral a used hereinafter includes intravenous, subcutaneous, intradermal, intramuscular, intraarterial injection, or by infusion techniques. The vaccine may be in the form of a single dose preparation or in multi-dose vials which can be used for mass vaccination programs. Suitable methods of preparing and using vaccines can be found in Remington's Pharmaceutical Sciences, Mack Publishing Co., Easton, Pa., Osol (ed.) (1980) and New Trends in Developments in Vaccines, Voller et al. (eds.), University Park Press, Baltimore, Md. (1978), incorporated by reference.

In some embodiments, a vaccine composition as disclosed herein may be administered parenterally in dosage unit formulations containing standard, well-known nontoxic physiologically acceptable carriers, adjuvants, and/or vehicles.

In some embodiments, the vaccine compositions may further comprise one or more adjuvants. An “adjuvant” is a substance that serves to enhance, accelerate, or prolong the antigen-specific immune response of an antigen when used in combination with specific vaccine antigens but do not stimulate an immune response when used alone. Suitable adjuvants include inorganic or organic adjuvants. Suitable inorganic adjuvants include, but ae not limited to, for example, an aluminium salt such as aluminum hydroxide gel (alum) or aluminum phosphate, but may also be a salt of calcium (particularly calcium carbonate), iron or zinc, or may be an insoluble suspension of acylated tyrosine, or acylated sugars, cationically or anionically derivitised polysaccharides or polyphospharanes. Other suitable adjuvants am known to one skilled in the art. Suitable Th1 adjuvant systems may also be used, and include, but am not limited to, for example, Monophosphoryl lipid A, other nontoxic derivatives of LPS, and combination of monophosphoryl lipid A, such as 3-de-O-acrylated monophosphorly lipid A (#D-MPL) together with an aluminium salt.

Other suitable examples of adjuvants include, but are not limited to, MF59, MPLA. Mycobacterium tuberculosis, Bordetella pertussis, bacterial lipopolysaccharides, aminoalkyl glucosamine phosphate compounds (AGP), or derivatives or analogs thereof, which ae available from Corixa (Hamilton, Mont.), and which are described in U.S. Pat. No. 6,113,918; e.g., 2-[(R)-3-Tetradecanoyloxytetradecanoylamino]ethyl, 2-Deoxy-4-O-phosphono-3-O—[(R)-3-tetradecanoyoxytetradecanoy 1]-2-[(R)-3-tetradecanoyoxytetradecanoylamino]-b-D-glucopyra noside, MPLTM. (3-O-deacylated monophosphoryl lipid A) (available from Corixa) described in U.S. Pat. No. 4,912,094, synthetic polynucleotides such as oligonucleotides containing a CpG motif (U.S. Pat. No. 6,207,646), COG-ODN (CpG oligodeoxynucleotides), polypeptides, saponins such as Quil A cr STIMULON.™. QS-21 (Antigenics, Framingham, Mass.), described in U.S. Pat. No. 5,057,540, a pertussis toxin (PT), or an E. coli heat-labile toxin (IT), particularly LT-K63, LT-R72, CT-5109, PT-K9/G129; see, e.g., International Patent Publication Nos. WO 93/13302 and WO 92/19265, cholera toxin (either in a wild-type or mutant form). Alternatively, various oil formulations such as stearyl tyrosine (ST, see U.S. Pat. No. 4,258,029), the dipeptide known as MDP, saponin, cholera toxin B subunit (CTB), a heat labile enterotoxin (LT) from E. coli (a genetically toxoided mutant LT has been developed), and Emulsomes (Phannos, LTD., Rehovot, Israel). Various cytokines and lymphokines are suitable for use as adjuvants. One such adjuvant is granulocyte-macrophage colony stimulating factor (GM-CSF), which has a nucleotide sequence as described in U.S. Pat. No. 5,078,996. The cytokine Interleukin-12 (IL-12) is another adjuvant which is described in U.S. Pat. No. 5,723,127. Other cytokines or lymphokines have been shown to have immune modulating activity, including, but not limited to, the interleukins 1-.alpha., 1-.beta., 2, 4, 5, 6, 7, 8, 1% 13, 14, 15, 16, 17 and 18, the interferons-.alpha., .beta., and .gamma., granulocyte colony stimulating factor, and the tumor necrosis factors .alpha. and .beta., and are suitable for use as adjuvants.

The vaccine compositions can be lyophilized to produce a vaccine against CHIKV in a dried form for ease in transportation and storage. Further, the vaccine may be prepared in the form of a mixed vaccine which contains the inactivated virus described herein and at least one other antigen as long as the added antigen does not interfere with the ability and/or efficacy of the vaccine, and as long as the added antigen does not induce additive or synergistic side effects and/or adverse reactions. The vaccine can be associated with chemical moieties which may improve the vaccine's solubility, absorption, biological half-life, etc. The moieties may alternatively decrease the toxicity of the vaccine, eliminate or attenuate any undesirable side effect of the vaccine, etc. Moieties capable of mediating such effects are disclosed in Remington's Pharmaceutical Sciences (1980). Procedures for coupling such moieties to a molecule are well known n the art.

The vaccine may be stored in a sealed vial, ampule or the like. The vaccines disclosed herein can generally be administered in the form of a spray for intranasal administration, or by nose drops, inhalants, swabs on tonsils, or a capsule, liquid, suspension or elixirs for oral administration. In the case where the vaccine is in a dried form, the vaccine is dissolved or suspended in sterilized distilled water before administration. Any inert carrier may be used, such as saline, phosphate buffered saline, or any such carrier in which the vaccine components have suitable solubility.

Vaccine compositions disclosed herein may include a carrier. If in a solution or a liquid aerosol suspension, suitable carriers can include, but are not limited to, salt solution, sucrose solution, or other pharmaceutically acceptable buffer solutions. Aerosol solutions may further comprise a surfactant.

Among the acceptable vehicles and solvents that may be used include water, Ringer's solution, and isotonic sodium chloride solution, including saline solutions buffered with phosphate, lactate, Tris and the like. In addition, sterile, fixed oils are conventionally employed as a solvent or suspending medium, including, but not limited to, for example, synthetic mono- or di-glycerides. In addition, fatty acids such as oleic acid find use in the preparation of injectables.

Injectable preparations, for example sterile injectable aqueous or oleaginous suspensions, are formulated according to the known art using suitable dispersing or wetting agents and suspending agents. The sterile injectable preparation are also a sterile injectable solution or suspension in a nontoxic parenterally acceptable diluent or solvent, for example, as a solution in 1,3-butanediol.

Some aspects and embodiments of the disclosure are illustrated by the following examples. These examples are provided to describe specific embodiments of the technology and do not limit the scope of the disclosure. It will be understood by those skilled in the art that the full scope of the disclosure is defined by the claims appending this specification, and any alterations, modifications, or equivalents of those claims.

### EXAMPLES

### Example 1. Passage and Derivation of CHIKV Strain 181/Clone 25

Chikungunya virus (CHIKV) was originally isolated from a human patient in Thailand (1962) and adapted to African green monkey kidney cells by passage (Harrison, V. R., et al. J. Immunol., 1971; 107:643-47). At the eleventh passage the CHIKV was inoculated into human MRC-5 and passaged 1B times with plaque selection of clone 25 (Levitt, N. H., et al. Vaccine, 1986; 4(3):157-621986). At passage 31 a master seed was manufactured, followed by passage 32 (working seed), and a vaccine lot at passage 33. Human clinical testing demonstrated immunogenicity and attenuation of the CHIKV 181/clone 25 strain (Edelman. R., et al. Am J Trap Med Hyg. 2000; 62(6):681-R5). For development of a new generation, purified-inactivated vaccine (PIV) CHIKV 181/done 25 was passaged in Vero cell. Vero passage-2 CHIKV has been deposited with the American Type Culture Collection at 10801 University Blvd. Manassas, Va. 20110-2209. USA, and given the ATCC Deposit Number PTA-126983. The ATCC deposit was made pursuant to the terms of the Budapest Treaty on the international recognition of the deposit of microorganisms for purposes of patent procedure. Table 1 lists titers of Vero passage-2 CHIKV. Yields of approximately 9 log 10 of CHIKV after two days in culture indicated that replication was sufficient for vaccine development.

TABLE 1
CHIKV (Vero p-2) replication in Vero cell cultures
at multiplicity of infection ratios of 0.1-0.001

| | | Lo2 CCID50/mL | | | Cyto]]atho2enic Effect | | |
|---|---|---|---|---|---|---|---|
| VIRUS | MOI | DAY-1 | DAY-2 | DAY-3 | DAY-1 | DAY-2 | DAY-3 |
| CHIKV | 0.1 | 9.0 | 9.2 | 7.5 | 1+ | 2+ | 4+ |
| P2-Vero | | | | | | | |
| CHIKV | 0.01 | 8.6 | 8.8 | 8.2 | 0 | 2+ | 4+ |
| P2-Vero | | | | | | | |
| CHIKV | 0.001 | 9.2 | 9.5 | 8.9 | 0 | 1+ | 4+ |
| P2-Vero | | | | | | | |

### Example 2 Purification of CHIKV Using CaptoCore Chromatography

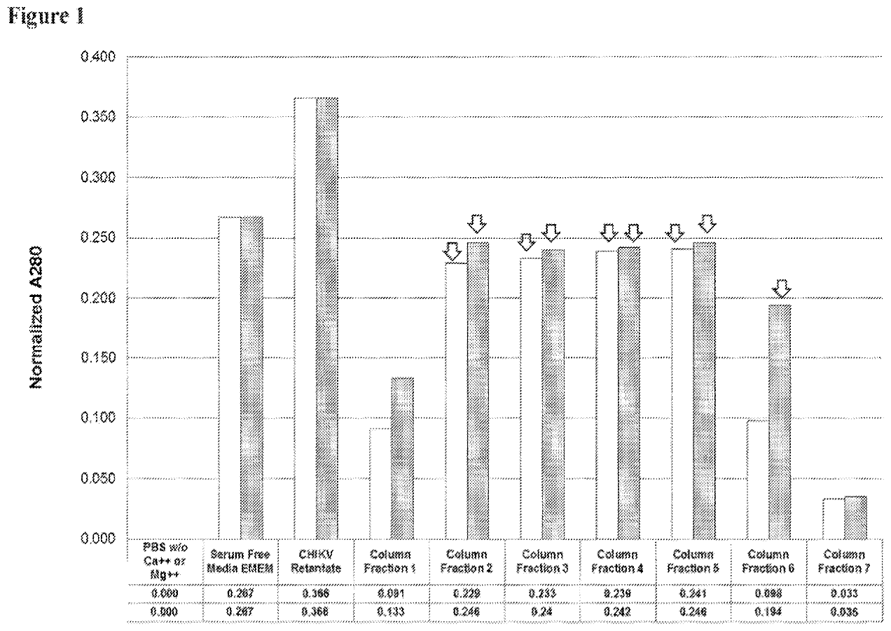

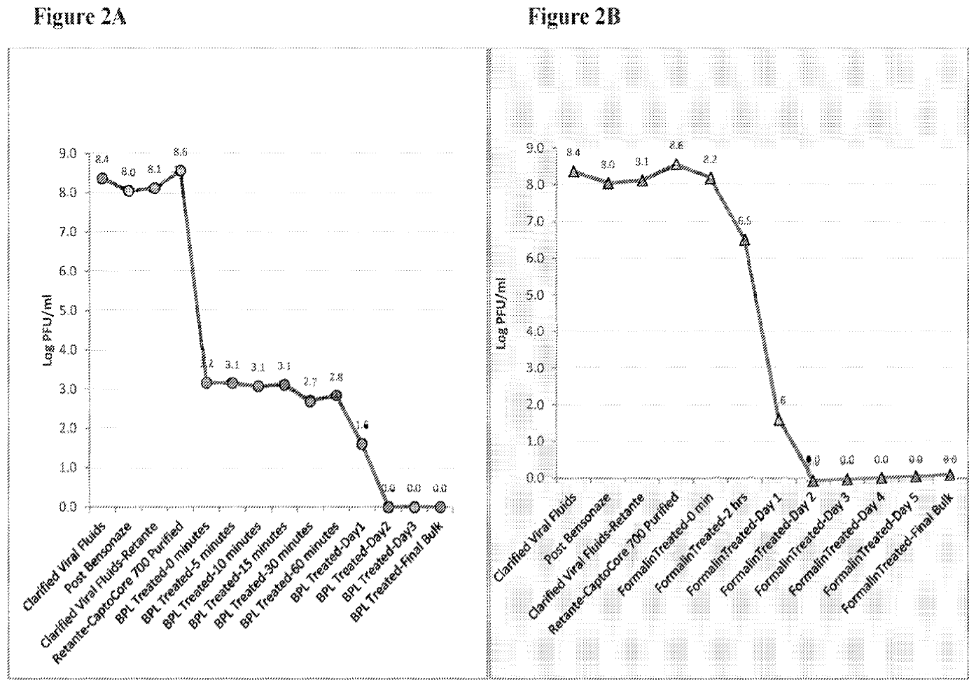

CHIKV supernatant fluids from Vero cell cultures were harvested at day 2 and clarified by low-speed centrifugation and filtration using a 0.45 micron filter. The clarified fluids were treated with 50,000 units/mL of benzonase for 2 hr at room temperature then concentrated by ultrafiltration using a 300 kD ultrafilter. Concentrated CHIKV was loaded onto a Captocore 700 chromatography column. Fractions were identified for collection by monitoring OD₂₈₀ readings. Column fractions 2-5 as shown in FIG. 1 were collected and pooled. FIG. 2 shows results from polyacrylamide electrophoresis of pre- and post-purification CHIKV after denaturation with SIDS.

### Example 3. Inactivation of CHIKV Using Formalin and Beta-Propiolactone (BPI)

Pooled column fractions were inactivated using 0.05% formalin or beta propiolactone (BPL) (from 0.025-1%) at 22-C. Samples of inactivated CHIKV were removed at intervals during inactivation as shown in FIGS. 2A and 2B. After 2 days, no live CHIKV could be detected by viral plaque assay. Additional days of inactivation (1-3 days) continued to ensure complete virus inactivation. Residual formalin in the final vaccine pool was neutralized by the addition of sodium metabisulfite.

### Example 4 Mouse Inoculation with CH/KV Purified-Inactivated Vaccine

Groups of 10 adult mice were vaccinated with vaccine doses ranging from 1,400 ng to 0.44 ng. Two doses were given (at 0 and 2 weeks) and sera were collected by terminal bleed at 4 weeks. Sera were tested for neutralizing antibody (MN50) in a plaque-reduction assay. Table 2 summarizes the results of the seroconversion rates and geometric mean neutralization titers for the groups of mice inoculated with formalin inactivated CHIKV. The effective immunizing dose 50% was calculated to be 37 ng. Table 3 summarizes the results of the seroconversion rates and geometric mean neutralization titers for the groups of mice inoculated with BPL inactivated CHIKV. The effective immunizing dose 50% was calculated to be 209 ng.

TABLE 2
Inoculation of adult mice with graded doses of HCHO inactivated
CHIKV vaccine

| CHIKVPIV | MN50 | MN50 | ED50 |
|---|---|---|---|
| (ng dose) | seroconversion | Geometric Mean | (ng) |
| 1,400 | 10/10 | 998 | 37 |
| 280 | 6/10 | 21 | |
| 56 | 7/10 | 59 | |
| 11 | 3/10 | 9 | |
| 2.2 | 1/10 | 4 | |
| 0.44 | 1/10 | 6 | |

TABLE 3
Inoculation of adult mice with graded doses of BPL inactivated
CHIKV vaccine

| CHIKVPIV | MN50 | MN50 | ED50 |
|---|---|---|---|
| (ng dose) | seroconversion | Geometric Mean | (ng) |
| 1,400 | 7/10 | 19 | 209 |
| 280 | 7/10 | 22 | |
| 56 | 1/10 | 5 | |
| 11 | 2/10 | 5 | |
| 2.2 | 3/10 | 8 | |
| 0.44 | 0/10 | 3 | |

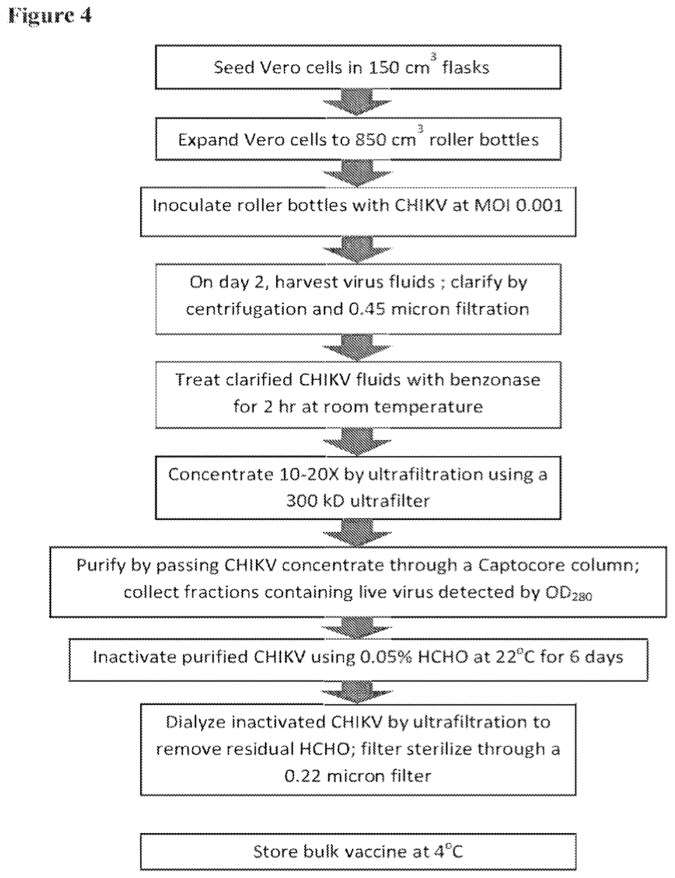

The production process shown in FIG. 4 based on the results of CHIKV production in Vero cell cultures, purification, and inactivation are suitable for further development and production of an inactivated CHIKV vaccine.

Based on the disclosure and the above data the compositions including Inactivated CHIKV demonstrate that the compositions are immunogenic and that vaccines comprising inactivated CHIKV are protective against infection with CHIKV.

### Example 5. CHIKV Purified-Inactivated Vaccine in Human Subjects

Healthy male and female volunteers are examined and screened using a panel of tests commonly used to identify acceptable participants. Volunteers are excluded if they have prior exposure t, aid possible existing immunity against, CHIKV. The volunteer demographics will be tracked and the groups will be constructed such that there will be no significant demographic differences between groups.

A standard randomized, single-blind inpatient clinical protocol will be used for all pilot studies. Each volunteer will be vaccinated with a determined dose of the candidate CHIKV PIV compositions. The volunteers will be monitored for adverse symptoms for a number of weeks (e.g., 2-4 weeks). Samples (e.g., blood, urine, saliva, etc.) will be collected from volunteers at regular intervals for standard medical analysis as well as for development of immune response (e.g., antibodies). Detection of anti-CHIKV antibodies will be identified using standard techniques known in the art (e.g., ELISA, modified ELISA). The candidate CHIKV PIV compositions will be identified as ineffective upon the identification of adverse clinical symptoms and/or failure to induce an immune response in the patients.

### INCORPORATION BY REFERENCE

All publications and patents mentioned herein are hereby incorporated by reference in their entirety as if each individual publication or patent was specifically and individually indicated to be incorporated by reference.

While specific aspects of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A method for immunizing a mammal against a CHIKV, wherein the method comprises administering to the mammal an effective amount of the a composition comprising a purified inactivated chikungunya virus (CHIKV).

2. A method of eliciting a protective immune response in a human against a chikungunya virus (CHIKV) infection comprising administering a composition comprising an effective amount of a purified inactivated chikungunya virus.

3. The method of claim 9, wherein the administering is via a route selected from intramuscular injection, intradermal injection, subcutaneous injection, intravenous injection, oral, or intranasal.

4. The method of claim 1, wherein the mammal is a human.

5. The method of claim 2, wherein the mammal is a human.

6. The method of claim 2, wherein the administering is via a route selected from intramuscular injection, intradermal injection, subcutaneous injection, intravenous injection, oral, or intranasal.

7. The method of claim 1, wherein the effective amount confers an antibody tier sufficient for broad-based sero-protection against all strains of purified inactivated chikungunya virus (CHIKV).

8. The method of claim 2, wherein the effective amount confers an antibody tier sufficient for broad-based sero-protection against all strains of purified inactivated chikungunya virus (CHIKV).

9. The method of claim 1, wherein the effective amount is about between about 56 and 1,400 ng purified inactivated chikungunya virus (CHIKV) per dose.

10. The method of claim 2, wherein the effective amount is about between about 56 and 1,400 ng purified inactivated chikungunya virus (CHIKV) per dose.

11. The method of claim 1, wherein the purified inactivated chikungunya virus (CHIKV) is the strain AF15561, La Reunion (LR) strain, or strain 181/clone 25 strain.

12. The method of claim 2, wherein the purified inactivated chikungunya virus (CHIKV) is the strain AF15561, La Reunion (LR) strain, or strain 181/clone 25 strain.

13. The method of claim 1, wherein the purified inactivated chikungunya virus (CHIKV) is inactivated by formalin or beta-propriolactone (PBL) treatment.

14. The method of claim 2, wherein the purified inactivated chikungunya virus (CHIKV) is inactivated by formalin or beta-propriolactone (PBL) treatment.

15. The method of claim 1, wherein the composition further comprises an adjuvant.

16. The method of claim 2, wherein the composition further comprises an adjuvant.
